SEQUENCE LISTING (24 sequences)

SEQ ID NO: 1 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 2 - protein, 4 aa, synthetic construct
TGGG

SEQ ID NO: 3 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 343 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6-11 - skipped (6 entries)

SEQ ID NO: 12 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 13 - protein, 100 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 14 - DNA, 1539 bp, Homo sapiens
  source 1..1539: mol_type = genomic DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 15 - DNA, 345 bp, Homo sapiens
  source 1..345: mol_type = genomic DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 16 - skipped

SEQ ID NO: 17 - protein, 4 aa, synthetic construct
GGGG

SEQ ID NO: 18 - protein, 5 aa, synthetic construct
TGGGG

SEQ ID NO: 19 - protein, 5 aa, synthetic construct
SGGGG

SEQ ID NO: 20 - skipped

SEQ ID NO: 21 - protein, 4 aa, synthetic construct
SGGG

SEQ ID NO: 22 - protein, 5 aa, synthetic construct
GGGGS

SEQ ID NO: 23 - DNA, 48 bp, synthetic construct
  source 1..48: mol_type = other DNA
cagacagacagacagacagacagacagacagacagacagacagacaga

SEQ ID NO: 24 - DNA, 72 bp, synthetic construct
  source 1..72: mol_type = other DNA
  misc_difference 1..72: note = This sequence may encompass 1-12 cagaca repeating units
cagacacagacacagacacagacacagacacagacacagacacagacacagacacagacacagacacagaca

(19) United States
(12) Patent Application Publication
(10) Pub. No.: US 2026/0098813 A1
(43) Pub. Date: Apr. 9, 2026

(54) IN VITRO CELL-BASED POTENCY ASSAY

(71) Applicant: Merck Sharp & Dohme LLC, Rahway, NJ (US)

(72) Inventors: Alicja Maria Cygan, Rahway, NJ (US); Joseph A. Sergi, Rahway, NJ (US); Asra Mirza, Rahway, NJ (US); Anka Ehrhardt, Rahway, NJ (US); Ping Han, Rahway, NJ (US); Junming Yie, Rahway, NJ (US)

(21) Appl. No.: 19/314,380

(22) Filed: Aug. 29, 2025

Related U.S. Application Data
(60) Provisional application No. 63/690,105, filed on Sep. 3, 2024.

(51) Int. Cl. G01N 21/76; C12Q 1/66; G01N 33/50; G01N 33/68

(52) U.S. Cl. CPC G01N 21/76; C12Q 1/66; G01N 33/5023; G01N 33/6863; G01N 2333/475; G01N 2333/71; G01N 2333/90241

(57) ABSTRACT

The present disclosure provides an in vitro cell-based potency assay to determine the relative potency of a composition, including a pharmaceutical composition, comprising ActRIIA-Fc fusion protein as compared to a reference sample.

### RELATED APPLICATIONS

This application claims the benefit of priority to U.S. Provisional Application Ser. No. 63/690,105, filed Sep. 3, 2024 (now pending). The specification of the foregoing application is hereby incorporated in its entirety.

### SEQUENCE LISTING

The instant application contains a Sequence Listing which has been submitted electronically in XML format and is hereby incorporated by reference in its entirety. Said XML copy, created on Jun. 30, 2025, is named 1848179-0002-174-101_SL.xml and is 20,198 bytes in size.

### FIELD OF THE INVENTION

The present disclosure provides an in vitro cell-based potency assay to determine the relative potency of sotatercept as compared to a reference sample.

### BACKGROUND OF THE INVENTION

Pulmonary arterial hypertension (PAH) is a severe and progressive disease characterized by high blood pressure in the arteries of the lungs due to vascular remodeling from cell proliferation. Symptoms experienced by PAH patients are mainly related to right heart dysfunction (Rosenkranz et al., Risk stratification and response to therapy in patients with pulmonary arterial hypertension and comorbidities: A COMPERA analysis, Journal of Heart and Lung Transplantation, Vol, 42, Issue 1, 102-114 (January 2023)). Patients are typically diagnosed in the prime of their lives, in their late forties, and are up to four times as likely to be women (Ruopp, N. F. et al., Diagnosis and Treatment of Pulmonary Arterial Hypertension A Review, JAMA, Vol. 327, No. 14 1379-1391 (2022)). PAH results in progressively debilitating symptoms and if left untreated, results in heart failure. There is a more than 43% 5-year mortality rate after diagnosis, and no substantial improvements have been made to improve survival within the past decade (Benza et al., An evaluation of long-term survival from time of diagnosis in pulmonary arterial hypertension from the reveal registry, Chest, Vol. 142, Issue 2, 448-456 (2012)). Current therapies focus on treating symptoms, but do not target the cellular level changes that contribute to PAH pathophysiology, so there is significant unmet need for new therapeutics for PAH (Mayeux et al., Management of Pulmonary Arterial Hypertension. In Current Cardiovascular Risk Reports, Current Cardiovascular Risk Reports 15:2 (2021)).

WINREVAIR™ (also known as sotatercept and MK-7962) is a transformational, first-in-class therapy for the treatment of PAH. It has the capacity to reverse the vascular remodeling observed in PAH due to its novel proposed mechanism of action, meaningfully improving patients exercise capacity and improving right heart function (Hoeper et al., Phase 3 Trial of Sotatercept for Treatment of Pulmonary Arterial Hypertension, New England Journal of Medicine (2023); Souza et al., Effects of sotatercept on haemodynamics and right heart function: analysis of the STELLAR trial. European Respiratory Journal, 62 (3), 2301107, (2023)). Sotatercept is a fusion protein comprised of the extracellular domain of human activin receptor IIA (ActRIIA) linked to the human IgG1 Fc domain. It functions as a ligand trap for select transforming growth factor beta (TGF-β) superfamily ligands, such as Activin A and growth differentiation factor 11 (GDF11), which are elevated in human PAH (Yung et al., ActRIIA-Fc rebalances activin/GDF versus BMP signaling in pulmonary hypertension, Sci. Transl. Med. 12, 5660 (2020)). These ligands preferentially activate the TGF-β effectors Smad2/3, via binding to activin receptor type II receptors (ActRIIA or ActRIIB) on the cell surface. PAH disease is driven by an imbalance in activin and growth differentiation factor (GDF) versus bone morphogenetic protein (BMP) signaling in pulmonary vascular smooth muscle and endothelial cells (Yung et al., ActRIIA-Fc rebalances activin/GDF versus BMP signaling in pulmonary hypertension, Sci. Transl. Med. 12, 5660 (2020)). Sotatercept rebalances this signaling, reversing the characteristic vascular remodeling seen in PAH.

For biologics such as sotatercept, validation of a cell culture-based functional cell-based assay (CBA) to determine the biological activity, or potency, of the biologic for release and stability is important. A validated potency assay is a regulatory requirement for the release of every lot of a biologic (International Conference on Harmonisation, 1999). Potency assays are invaluable tools used to demonstrate lot stability, lot-to-lot consistency, assess product comparability after manufacturing changes, and to evaluate product quality attributes. Regulatory expectations for a potency assay include that they provide quantitative data, meet pre-defined acceptance and/or rejection criteria, include appropriate controls and reference materials, are stability-indicating and sensitive to structural changes, are reflective of the mechanism of action of a product, and are well-controlled and reproducible (US Department of Health and Human Services, 2011). From an industry perspective, potency assays should also be operationally simple to perform in a quality control (QC) lab, so as to minimize potential for assay failures due to operator error and thus prevent delay of drug release to patients.

### BRIEF SUMMARY OF THE INVENTION

Provided herein are methods for determining the relative in vitro potency of a composition comprising an ActRIIA-Fc fusion protein.

In certain embodiments, the methods for determining the relative in vitro potency of an ActRIIA-Fc fusion protein or a composition comprising the ActRIIA-Fc fusion protein described herein comprise:
(i) adding a TGF-β super family ligand to a first portion and a second portion of a cell suspension comprising cells that endogenously express the receptors required for TGF-β super family ligand activation, wherein the cells are engineered to stably express a firefly luciferase (FFLuc) reporter gene, wherein the TGF-β super family ligand is selected from Activin A, Activin B and GDF11;
(ii) adding a test sample comprising a known amount of the ActRIIA-Fc fusion protein to the first portion of the cell suspension to make a test mixture;
(iii) adding a reference sample comprising the ActRIIA-Fc fusion protein to the second portion of the cell suspension to make a reference mixture;
(iv) adding a chemiluminescent substrate solution to the test mixture and the reference mixture;
(v) measuring the luminescence of the test mixture and the reference mixture; and
(vi) determining the relative potency by comparing the luminescence of the test mixture and the reference mixture.

In certain embodiments, the first portion and the second portion of the cell suspension are incubated for at least 10 minutes after adding the TGF-β super family ligand. In certain embodiments, the first portion and the second portion of the cell suspension are incubated between 1-4 hours after adding the TGF-β super family ligand.

In certain embodiments, the first portion and the second portion of the cell suspension are incubated at a temperature between 30-40° C. In certain embodiments, the first portion and the second portion of the cell suspension are incubated at a temperature of about 37° C. and about 5% CO₂.

In certain embodiments of the methods for determining the relative in vitro potency of an ActRIIA-Fc fusion protein or a composition comprising the ActRIIA-Fc fusion protein described herein, the test sample is added to the first portion of the cell suspension at the same time as the TGF-β super family ligand.

In certain embodiments of the methods for determining the relative in vitro potency of an ActRIIA-Fc fusion protein or a composition comprising the ActRIIA-Fc fusion protein described herein, the reference sample is added to the second portion of the cell suspension at the same time as the TGF-β super family ligand.

In certain embodiments of the methods for determining the relative in vitro potency of an ActRIIA-Fc fusion protein or a composition comprising the ActRIIA-Fc fusion protein described herein, before step (v), the reference mixture and the test mixture are incubated overnight. In certain embodiments, before step (v), the reference mixture and the test mixture are incubated for about 24 hours.

In certain embodiments of the methods for determining the relative in vitro potency of an ActRIIA-Fc fusion protein or a composition comprising the ActRIIA-Fc fusion protein described herein, the reference mixture and the test mixture are incubated at a temperature between 30-40° C. In certain embodiments, the reference mixture and the test mixture are incubated at about 37° C. and about 5% CO₂.

In certain embodiments of the methods for determining the relative in vitro potency of an ActRIIA-Fc fusion protein or a composition comprising the ActRIIA-Fc fusion protein described herein, the chemoluminescent substrate solution is ONE-Glo™ Luciferase Assay System.

In certain embodiments of the methods for determining the relative in vitro potency of an ActRIIA-Fc fusion protein or a composition comprising the ActRIIA-Fc fusion protein described herein, the cell suspension further comprises Opti-MEM medium.

In certain embodiments, the cell suspension further comprises fetal bovine serum.

In certain embodiments of the methods for determining the relative in vitro potency of an ActRIIA-Fc fusion protein or a composition comprising the ActRIIA-Fc fusion protein described herein, further comprise seeding the cells on a cell culture plate comprising at least 12, 24, 48, 96 or 384 wells prior to adding the TGF-β super family ligand to the first portion and the second portion of the cell suspension.

In certain embodiments, the cell culture plate is seeded with between 5,000 and 50,000 cells per well. In certain embodiments, the cell culture plate is seeded with between 15,000 and 25,000 cells per well. In certain embodiments, the cell culture plate is seeded with about 20,000 cells per well. In certain embodiments, the cell culture plate comprises 96 wells and wherein the cell culture plate is seeded with about 20,000 cells per well.

In certain embodiments of the methods for determining the relative in vitro potency of an ActRIIA-Fc fusion protein or a composition comprising the ActRIIA-Fc fusion protein described herein, further comprise generating a dose response curve for the test mixture and the reference mixture and determining the reference light unit (RLU) of the test mixture and the reference mixture.

In certain embodiments of the methods for determining the relative in vitro potency of an ActRIIA-Fc fusion protein or a composition comprising the ActRIIA-Fc fusion protein described herein, the relative potency is calculated as a percentage of the reference standard RLU using the formula:

$$\% Relative potency = ( Creference / Csample ( or control ) ) \times RS assigned potency per COA$$

In certain embodiments of the methods for determining the relative in vitro potency of an ActRIIA-Fc fusion protein or a composition comprising the ActRIIA-Fc fusion protein described herein, the reporter gene is under the control of a (CAGA)₁₂ promoter (SEQ ID NO: 23) or a CAGACA promoter. In certain embodiments, the reporter gene is under the control of a (CAGA)₁₂ promoter (SEQ ID NO: 23).

In certain embodiments of the methods for determining the relative in vitro potency of an ActRIIA-Fc fusion protein or a composition comprising the ActRIIA-Fc fusion protein described herein, the TGF-β super family ligand is Activin A.

In certain embodiments of the methods for determining the relative in vitro potency of an ActRIIA-Fc fusion protein or a composition comprising the ActRIIA-Fc fusion protein described herein, the ActRIIA-Fc fusion protein comprises an amino acid sequence that is at least 90% identical to SEQ ID NO: 1. In certain embodiments, the ActRIIA-Fc fusion protein comprises an amino acid sequence that is at least 95% identical to SEQ ID NO: 1. In certain embodiments, the ActRIIA-Fc fusion protein comprises an amino acid sequence that is at least 99% identical to SEQ ID NO: 1. In certain embodiments, the ActRIIA-Fc fusion protein comprises the amino acid sequence of SEQ ID NO: 1. In certain embodiments, the ActRIIA-Fc fusion protein comprises an amino acid sequence that is at least 90% identical to SEQ ID NO: 4. In certain embodiments, the ActRIIA-Fc fusion protein comprises an amino acid sequence that is at least 95% identical to SEQ ID NO: 4. In certain embodiments, the ActRIIA-Fc fusion protein comprises an amino acid sequence that is at least 99% identical to SEQ ID NO: 4. In certain embodiments, the ActRIIA-Fc fusion protein comprises the amino acid sequence of SEQ ID NO: 4. In certain embodiments, the ActRIIA-Fc fusion protein comprises an amino acid sequences that is at least 90% identical to SEQ ID NO: 5. In certain embodiments, the ActRIIA-Fc fusion protein comprises an amino acid sequence that is at least 95% identical to SEQ ID NO: 5. In certain embodiments, the ActRIIA-Fc fusion protein comprises an amino acid sequence that is at least 99% identical to SEQ ID NO: 5. In certain embodiments, the ActRIIA-Fc fusion protein comprises the amino acid sequence of SEQ ID NO: 5.

In certain embodiments of the methods for determining the relative in vitro potency of an ActRIIA-Fc fusion protein or a composition comprising the ActRIIA-Fc fusion protein described herein, the ActRIIA-Fc fusion protein is sotatercept.

Also described herein are release assays for testing a batch of a pharmaceutical composition comprising an ActRIIA-Fc fusion protein, comprising
(i) determining the relative in vitro potency of a test mixture of the pharmaceutical composition from the batch according to any of the methods described herein; and
(ii) releasing the batch for in vivo use if the relative in vitro potency value determined in step (i) falls within a range of acceptable values.

In certain embodiments, the relative in vitro potency value is calculated by generating a dose response curve for the test sample and the reference sample and determining the EC50 of the test sample and reference sample.

In certain embodiments of the release assays for testing a batch of a pharmaceutical composition comprising an ActRIIA-Fc fusion protein described herein, the relative in vitro potency value is calculated using the formula:

$$( IC 50 reference standard / IC 50 test sample ) * 100.$$

In certain embodiments of the release assays for testing a batch of a pharmaceutical composition comprising an ActRIIA-Fc fusion protein described herein, the acceptable relative in vitro potency value is between 50% and 200%. In certain embodiments, the acceptable relative in vitro potency value is between 70% and 135%.

In certain embodiments of the release assays for testing a batch of a pharmaceutical composition comprising an ActRIIA-Fc fusion protein described herein, the ActRIIA-Fc fusion protein comprises an amino acid sequence that is at least 90% identical to SEQ ID NO: 1. In certain embodiments, the ActRIIA-Fc fusion protein comprises an amino acid sequence that is at least 95% identical to SEQ ID NO: 1. In certain embodiments, the ActRIIA-Fc fusion protein comprises an amino acid sequence that is at least 99% identical to SEQ ID NO: 1. In certain embodiments, the ActRIIA-Fc fusion protein comprises the amino acid sequence of SEQ ID NO: 1. In certain embodiments, the ActRIIA-Fc fusion protein comprises an amino acid sequence that is at least 90% identical to SEQ ID NO: 4. In certain embodiments, the ActRIIA-Fc fusion protein comprises an amino acid sequence that is at least 95% identical to SEQ ID NO: 4. In certain embodiments, the ActRIIA-Fc fusion protein comprises an amino acid sequence that is at least 99% identical to SEQ ID NO: 4. In certain embodiments, the ActRIIA-Fc fusion protein comprises the amino acid sequence of SEQ ID NO: 4. In certain embodiments, the ActRIIA-Fc fusion protein comprises an amino acid sequence that is at least 90% identical to SEQ ID NO: 5. In certain embodiments, the ActRIIA-Fc fusion protein comprises an amino acid sequence that is at least 95% identical to SEQ ID NO: 5. In certain embodiments, the ActRIIA-Fc fusion protein comprises an amino acid sequence that is at least 99% identical to SEQ ID NO: 5. In certain embodiments, the ActRIIA-Fc fusion protein comprises the amino acid sequence of SEQ ID NO: 5. In certain embodiments, the ActRIIA-Fc fusion protein comprises sotatercept.

Also provided herein are methods for determining the relative in vitro potency of a composition comprising sotatercept.

In certain embodiments the methods for determining the relative in vitro potency of sotatercept or a composition comprising sotatercept, comprise (i) preparing a cell suspension wherein the cell suspension comprises cells that endogenously express the receptors required for Activin A activation and are engineered to stably express a firefly luciferase (FFLuc) reporter gene under a (CAGA)₁₂ promoter (SEQ ID NO: 23) (Smad2/3 reporter cell line); (ii) adding Activin A to a first portion and a second portion of the cell suspension; (iii) adding a test sample comprising sotatercept to the first portion of the mixture of the cell suspension and Activin A to make a test mixture; (iv) adding a reference sample comprising sotatercept to a second portion of the mixture of the cell suspension and Activin A to make a reference mixture; (v) adding a chemiluminescent substrate solution to the test mixture and the reference mixture; (vi) measuring the luminescence of the test mixture and the reference mixture; and (vii) comparing the luminescence of test mixture and the reference mixture.

In certain embodiments, between step (i) and (ii), the cell suspension is incubated for at least 10 minutes before adding the Activin A. In certain embodiments, between step (i) and (ii), the cell suspension is incubated between 1-4 hours before adding the Activin A.

In certain embodiments the methods for determining the relative in vitro potency of sotatercept or a composition comprising sotatercept, the cell suspension is incubated at a temperature between 30-40° C. In certain embodiments the methods for determining the relative in vitro potency of sotatercept or a composition comprising sotatercept the cell suspension is incubated at about 37° C. and about 5% CO₂.

In certain embodiments the methods for determining the relative in vitro potency of sotatercept or a composition comprising sotatercept, the test sample comprising sotatercept and the Activin A, is added to the first portion of the cell suspension at the same time.

In certain embodiments of the methods for determining the relative in vitro potency of sotatercept or a composition comprising sotatercept, the reference sample comprising sotatercept and the Activin A is added to the second portion of the cell suspension at the same time.

In certain embodiments of the methods for determining the relative in vitro potency of sotatercept or a composition comprising sotatercept, before step (v), the reference mixture and the test mixture are incubated overnight. In certain embodiments of the methods for determining the relative in vitro potency of sotatercept or a composition comprising sotatercept, before step (v), the reference mixture and the test mixture are incubated for about 24 hours.

In certain embodiments of the methods for determining the relative in vitro potency of sotatercept or a composition comprising sotatercept, before step (v), the reference mixture and the test mixture are incubated at a temperature between 30-40° C.

In certain embodiments of the methods for determining the relative in vitro potency of sotatercept or a composition comprising sotatercept, before step (v), the reference mixture and the test mixture are incubated at about 37° C. and about 5% CO₂.

In certain embodiments of the methods for determining the relative in vitro potency of sotatercept or a composition comprising sotatercept, the chemoluminescent substrate solution is ONE-Glo™ Luciferase Assay System.

In certain embodiments of the methods for determining the relative in vitro potency of sotatercept or a composition comprising sotatercept, the cell suspension further comprises Opti-MEM medium. In certain embodiments of the methods for determining the relative in vitro potency of sotatercept or a composition comprising sotatercept, the cell suspension further comprises fetal bovine serum.

In certain embodiments of the methods for determining the relative in vitro potency of sotatercept or a composition comprising sotatercept, cells are seeded on a cell culture plate comprising at least 12, 24, 48, 96 or 384 wells prior to adding Activin A to the cell suspension.

In certain embodiments of the methods for determining the relative in vitro potency of sotatercept or a composition comprising sotatercept, the cell culture plate is seeded with between 5,000 and 50,000 cells per well. In certain embodiments of the methods for determining the relative in vitro potency of sotatercept or a composition comprising sotatercept, the cell culture plate is seeded with between 15,000 and 25,000 cells per well.

In certain embodiments of the methods for determining the relative in vitro potency of sotatercept or a composition comprising sotatercept, the cell culture plate is seeded with about 20,000 cells per well.

In certain embodiments of the methods for determining the relative in vitro potency of sotatercept or a composition comprising sotatercept, the cell culture plate comprises 96 wells and wherein the cell culture plate is seeded with about 20,000 cells per well.

In certain embodiments of the methods for determining the relative in vitro potency of sotatercept or a composition comprising sotatercept, the method further comprises generating a dose response curve for the test mixture and the reference mixture and determining the reference light unit (RLU) of the test mixture and the reference mixture.

In certain embodiments of the methods for determining the relative in vitro potency of sotatercept or a composition comprising sotatercept, the relative potency is calculated as a percentage of the reference standard RLU using the formula:

$$\% Relative potency = ( Creference / Csample ( or control ) ) \times RS assigned potency per COA$$

Also described herein are processes for releasing or accepting a batch of a pharmaceutical composition comprising an ActRIIA-Fc fusion protein, comprising: (i) determining the relative in vitro potency of a test mixture of the pharmaceutical composition from the batch according to any of the methods described herein; and (ii) releasing further pharmaceutical compositions from the batch for in vivo use if the results of step (i) indicate an acceptable relative in vitro potency value.

In certain embodiments of the processes described herein, the relative in vitro potency value is calculated by generating a dose response curve for the test sample and the reference sample and determining the EC50 of the test sample and reference sample.

In certain embodiments of the processes described herein, the relative in vitro potency value is calculated using the formula:

$$( IC 50 reference standard / IC 50 test sample ) * 100$$

In certain embodiments of the processes described herein, the acceptable relative in vitro potency value is calculated to be between 50% and 200%. In certain embodiments of the processes described herein, the acceptable relative in vitro potency value is calculated to be between 70% and 135%.

In certain embodiments of the processes described herein, the ActRIIA-Fc fusion protein comprises an amino acid sequence that is at least 90% identical to SEQ ID NO: 1. In certain embodiments, the ActRIIA-Fc fusion protein comprises an amino acid sequence that is at least 95% identical to SEQ ID NO: 1. In certain embodiments, the ActRIIA-Fc fusion protein comprises an amino acid sequence that is at least 99% identical to SEQ ID NO: 1. In certain embodiments, the ActRIIA-Fc fusion protein comprises the amino acid sequence of SEQ ID NO: 1. In certain embodiments, the ActRIIA-Fc fusion protein comprises an amino acid sequence that is at least 90% identical to SEQ ID NO: 4. In certain embodiments, the ActRIIA-Fc fusion protein comprises an amino acid sequence that is at least 95% identical to SEQ ID NO: 4. In certain embodiments, the ActRIIA-Fc fusion protein comprises an amino acid sequence that is at least 99% identical to SEQ ID NO: 4. In certain embodiments, the ActRIIA-Fc fusion protein comprises the amino acid sequence of SEQ ID NO: 4. In certain embodiments, the ActRIIA-Fc fusion protein comprises an amino acid sequences that is at least 90% identical to SEQ ID NO: 5. In certain embodiments, the ActRIIA-Fc fusion protein comprises an amino acid sequence that is at least 95% identical to SEQ ID NO: 5. In certain embodiments, the ActRIIA-Fc fusion protein comprises an amino acid sequence that is at least 99% identical to SEQ ID NO: 5. In certain embodiments, the ActRIIA-Fc fusion protein comprises the amino acid sequence of SEQ ID NO: 5.

In certain embodiments of the methods for determining the relative in vitro potency of an ActRIIA-Fc fusion protein or a composition comprising the ActRIIA-Fc fusion protein described herein, the ActRIIA-Fc fusion protein comprises sotatercept.

### BRIEF DESCRIPTION OF THE DRAWINGS

FIG. 1 depicts the principles of the cell-based assays described herein. Smad2/3 signaling is activated in this cell line upon addition of exogenous Activin A ligand and is detected by the expression of luciferase. The ability of sotatercept to trap the exogenous ligand and subsequently prevent the induction of the Smad2/3 signaling is determined by detecting the inhibition of the luminescence signal.

FIG. 2 shows a representative sotatercept cell-based assay dose response curve. The luminescence signal, proportional to inhibition of Smad2/3 signaling at different dilutions of sotatercept, is quantified using a luminescence plate reader. Potency is expressed as a percentage relative to reference material.

FIG. 3 sets forth a general schematic of the in vitro cell-based potency assay.

### DETAILED DESCRIPTION OF THE INVENTION

### Definitions and Abbreviations

As used throughout the specification and appended claims, the following abbreviations apply:
C: the inflection point of the 4PL curve fit or IC50 (ng/ml)
CPW: Cells per well
FBS: Fetal Bovine Serum
QC: Quality control
RLU: Reference Light Unit
RS: Reference Standard

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Reference to “or” indicates either or both possibilities unless the context clearly dictates one of the indicated possibilities. In some cases, “and/or” was employed to highlight either or both possibilities.

As used herein, including the appended claims, the singular forms of words such as “a,” “an,” and “the,” include their corresponding plural references unless the context clearly dictates otherwise.

The term “about”, when modifying the quantity of a substance, the pH of a solution/formulation, or the value of a parameter characterizing a step in a method, or the like refers to variant in the numerical quantity that can occur, for example, through typical measuring, handling and sampling procedures involved in the preparation, characterization and/or use of the substance or composition; through inadvertent error in these procedures, through differences in the manufacture, source or purity of the ingredients employed to make or use the compositions or carryout the procedures and the like. In certain embodiments, “about” can mean a variation of greater or lesser than the value or range of values stated by 10 percent, e.g., ±0.2%, 0.5%, 1%, 2%, 3%, 4%, 5%, or 10%. Each value or range of values preceded by the term “about” is also intended to encompass the embodiment of the stated absolute value or range of values.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified composition or method.

“Relative in vitro potency” or “relative potency” as used herein is the comparison of the potency of a test sample to the potency of a reference standard. For example, when potency is determined at the EC50, the relative potency of the test sample is calculated as a percentage of the reference standard EC50 as follows:

$$( EC 50 reference standard / EC 50 test sample ) * 100$$

“Dose response curves” generated using the cell-based potency assay described herein refer to the dose of an ActRIIA-Fc fusion protein, such as sotatercept, and the response refers to the measured parameter (e.g., expression) that corresponds to such dose.

“Test Sample” or “Test Article” as used herein refers to an aliquot of material obtained from a source of interest, such as, for example, a pharmaceutical composition comprising sotatercept. Analysis of the test samples by the assay described herein provides information about the relative potency of the samples. In embodiments of the methods described herein, the test sample is analyzed at a fraction of full strength, e.g., after dilution.

“Reference Sample” or “Reference Standard” as used herein describes a standard or control sample relative to which a test sample is compared. As used herein, the reference standard of a composition comprising an ActRIIA-Fc fusion protein, such as sotatercept, designated as having a potency of 100%, and is used to calculate the relative potency of a test article or test sample. As a non-limiting example, a reference sample may be a sample which has been shown to exhibit good therapeutic effect in vivo. Typically, as understood by those skilled in the art, a reference sample is determined or characterized under comparable conditions or circumstances to those under assessment (e.g., to the test sample). In embodiments of the methods described herein, the reference sample is analyzed at a fraction of full strength, e.g., after dilution. As a non-limiting example, the reference sample (and the test sample) may be analyzed using a 2, 3, 3.5, 3.2, 5, 10 or 15-fold serial dilution.

“Plate” or “Cell plate” or “Cell culture plate” means multiwell plate(s). In an embodiment of the invention, “plate(s)” “cell plate(s)” or “cell culture plate(s)” means multiwell plate(s) that are manufactured with 6, 12, 24, 48, 96, 384 or 1536 sample wells in each plate. In another embodiment, plate(s) are multiwell plate(s) with 24 sample wells or more. In another embodiment, plate(s) are multiwell plate(s) with 48 sample wells or more. In another embodiment, plate(s) are multiwell plate(s) with 96 sample wells or more. In another embodiment, plate(s) are multiwell plate(s) with 384 sample wells or more. In another embodiment, plate(s) are multiwell plate(s) with 1536 sample wells or more. In another embodiment, plate(s) are 96 sample well plate(s). In another embodiment, plate(s) are 384 sample well plate(s). In another embodiment, plate(s) are 1536 sample well plate(s).

The term “seeded” or “seeding” means the addition of cells to a plate with appropriate growth media. The term “seeding density” as used herein is the concentration or number of cells that are added per each well of a multiwell plate to form a monolayer. In some embodiments the seeding density is between about 5×10₄ cells per well to 1.2×10⁵ cells per well, when the plate is a multiwell plate with 96 wells and which does not contain a coating. In another embodiment, the seeding density is between 1.0×10⁵ cells per well and about 1.2×10⁵ cells per well, when the plate is a multiwell plate with 96 wells and which does not contain a coating. In a further embodiment, the seeding density is about 1.2×10⁵ cells per well, when the plate is a multiwell plate with 96 wells and which does not contain a coating.

In another embodiment, the seeding density is up to 35,000 cells per well, when the plate is a multiwell plate with 96 wells and contains a coating. In another embodiment, the seeding density is between 15,000 and 35,000 cells per well, when the plate is a multiwell plate with 96 wells and contains a coating. In a further embodiment, the seeding density is between 20,000 cells per well and 30,000 cells per well, when the plate is a multiwell plate with 96 wells and contains a coating. In another embodiment, the seeding density is about 20,000 cells per well, when the plate is a multiwell plate with 96 wells and contains a coating. In another embodiment, the seeding density is about 30,000 cells per well, when the plate is a multiwell plate with 96 wells and contains a coating.

### ActRIIA-Fc Fusion Proteins

Described herein are methods for determining the relative in vitro potency of an ActRIIA-Fc fusion protein or a composition comprising an ActRIIA-Fc fusion protein. An ActRIIA fusion protein is an ActRIIA polypeptide that is linked to an Fc domain via a linker. As used herein, the term “ActRIIA” refers to a family of activin receptor type IIA (ActRIIA) proteins, and/or variants thereof. The ActRIIA polypeptides can be derived from any species and include variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term ActRIIA polypeptide includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the disclosure as well as in International Patent Application Publication Nos. WO 2006/012627, WO 2007/062188, WO 2008/097541, WO 2010/151426, and WO 2011/020045, which are incorporated herein by reference in their entirety.

In certain embodiments, the ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in SEQ ID NO: 1. In other embodiments, the ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in SEQ ID NO: 12. In other embodiments, the ActRIIA polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in SEQ ID NO: 13.

In some embodiments, the ActRIIA polypeptide comprises an amino acid sequence that is at least 90%, 91%, 92%, 93%, 94%, 95%, 96%, 97%, 98%, 99%, or 100% identical to an amino acid sequence corresponding to residues 30-110 of SEQ ID NO: 12. In some embodiments, the ActRIIA polypeptide comprises an amino acid sequence that is at least 90%, 91%, 92%, 93%, 94%, 95%, 96%, 97%, 98%, 99%, or 100% identical to an amino acid sequence corresponding to residues 21-135 of SEQ ID NO: 12.

Numbering of amino acids for all ActRIIA-related polypeptides described herein is based on the numbering of the human ActRIIA precursor protein sequence provided below (SEQ ID NO: 12), unless specifically designated otherwise.

| (SEQ ID NO: 12) | |
|---|---|
| 1 | MGAAAKLAFA VFLISCSSGA ILGRSETQEC LFFNANWEKD RTNQTGVEPC |
| 51 | YGDKDKRRHC FATWKNISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFORD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A nucleic acid sequence encoding the human ActRIIA precursor protein (SEQ ID NO: 12) is shown below (SEQ ID NO: 14), as follows nucleotides 159-1700 of Genbank Reference Sequence NM_001616.4. The signal sequence is underlined.

| (SEQ ID NO: 14) | |
|---|---|
| 1 | atgggagctg ctgcaaagtt ggcgtttgcc gtctttctta tctcctgttc |
| 51 | ttcaggtgct atacttggta gatcagaaac tcaggagtgt cttttcttta |
| 101 | atgctaattg ggaaaaagac agaaccaatc aaactggtgt tgaaccgtgt |
| 151 | tatggtgaca aagataaacg gcggcattgt tttgctacct ggaagaatat |
| 201 | ttctggttcc attgaaatag tgaaacaagg ttgttggctg gatgatatca |
| 251 | actgctatga caggactgat tgtgtagaaa aaaaagacag ccctgaagta |
| 301 | tatttttgtt gctgtgaggg caatatgtgt aatgaaaagt tttcttattt |
| 351 | tccggagatg gaagtcacac agcccacttc aaatccagtt acacctaagc |
| 401 | caccctatta caacatcctg ctctattcct tggtgccact tatgttaatt |
| 451 | gcggggattg tcatttgtgc attttgggtg tacaggcatc acaagatggc |
| 501 | ctaccctcct gtacttgttc caactcaaga cccaggacca cccccacctt |
| 551 | ctccattact aggtttgaaa ccactgcagt tattagaagt gaaagcaagg |
| 601 | ggaagatttg gttgtgtctg gaaagcccag ttgcttaacg aatatgtggc |
| 651 | tgtcaaaata tttccaatac aggacaaaca gtcatggcaa aatgaatacg |
| 701 | aagtctacag tttgcctgga atgaagcatg agaacatatt acagttcatt |
| 751 | ggtgcagaaa aacgaggcac cagtgttgat gtggatcttt ggctgatcac |
| 801 | agcatttcat gaaaagggtt cactatcaga ctttcttaag gctaatgtgg |
| 851 | tctcttggaa tgaactgtgt catattgcag aaaccatggc tagaggattg |
| 901 | gcatatttac atgaggatat acctggccta aaagatggcc acaaacctgc |
| 951 | catatctcac agggacatca aaagtaaaaa tgtgctgttg aaaaacaacc |
| 1001 | tgacagcttg cattgctgac tttgggttgg ccttaaaatt tgaggctggc |
| 1051 | aagtctgcag gcgataccca tggacaggtt ggtacccgga ggtacatggc |
| 1101 | tccagaggta ttagagggtg ctataaactt ccaaagggat gcatttttga |
| 1151 | ggatagatat gtatgccatg ggattagtcc tatgggaact ggcttctcgc |
| 1201 | tgtactgctg cagatggacc tgtagatgaa tacatgttgc catttgagga |
| 1251 | ggaaattggc cagcatccat ctcttgaaga catgcaggaa gttgttgtgc |
| 1301 | ataaaaaaaa gaggcctgtt ttaagagatt attggcagaa acatgctgga |
| 1351 | atggcaatgc tctgtgaaac cattgaagaa tgttgggatc acgacgcaga |
| 1401 | agccaggtta tcagctggat gtgtaggtga aagaattacc cagatgcaga |
| 1451 | gactaacaaa tattattacc acagaggaca ttgtaacagt ggtcacaatg |
| 1501 | gtgacaaatg ttgactttcc toccaaagaa tctagtcta |

A nucleic acid sequence encoding the processed soluble (extracellular) human ActRIIA polypeptide (SEQ ID NO: 1) is as follows:

| (SEQ ID NO: 15) | |
|---|---|
| 1 | atacttggta gatcagaaac tcaggagtgt cttttcttta atgctaattg |
| 51 | ggaaaaagac agaaccaatc aaactggtgt tgaaccgtgt tatggtgaca |
| 101 | aagataaacg gcggcattgt tttgctacct ggaagaatat ttctggttcc |
| 151 | attgaaatag tgaaacaagg ttgttggctg gatgatatca actgctatga |
| 201 | caggactgat tgtgtagaaa aaaaagacag ccctgaagta tatttttgtt |
| 251 | gctgtgaggg caatatgtgt aatgaaaagt tttcttattt tccggagatg |
| 301 | gaagtcacac agcccacttc aaatccagtt acacctaagc caccc |

The ActRIIA polypeptide sequence comprises the sequence disclosed at accession number UniProtKB/Swiss-Prot P27037.1, and/or variants thereof. In some embodiments, the term “wild-type ActRIIA polypeptide” refers to the extracellular domain of ActRIIA, amino acids 1 to 135 (with signal sequence), or amino acids 20 through 135 of SEQ ID NO: 1 (without signal sequence)

In certain embodiments, the ActRIIA-Fc fusion protein is a fusion protein further comprising an Fc domain of an immunoglobulin. In some embodiments, the Fc domain of the immunoglobulin is an Fc domain of an IgG1 immunoglobulin. In some embodiments, the Fc domain comprises an amino acid sequence that is at least 90%, 91%, 92%, 93%, 94%, 95%, 96%, 97%, 98%, 99%, or 100% identical to the amino acid sequence of SEQ ID NO: 3.

In some embodiments, the Fc fusion protein further comprises a linker domain positioned between the ActRIIA polypeptide domain and the Fc domain of the immunoglobulin. In some embodiments, the linker domain is selected from the group consisting of: TGGG (SEQ ID NO: 2), TGGGG (SEQ ID NO: 18), SGGGG (SEQ ID NO: 19), GGGGS (SEQ ID NO: 22), GGG, GGGG (SEQ ID NO: 17), and SGGG (SEQ ID NO: 21). In some embodiments, the ActRIIA-Fc fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 4. In some embodiments, the ActRIIA-Fc fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 5.

### Sotatercept

Sotatercept is a first-in-class fusion protein comprised of the extracellular domain of human ActRIIA linked to the fragment crystallizable domain of human immunoglobulin G. Sotatercept, an activin signaling inhibitor, acts as a selective ligand trap for pro-proliferative activins and related growth differentiation factors; this restores the balance between the growth-promoting and growth-inhibiting signaling pathways. In 2024, the US Food and Drug Administration approved WINREVAIR® (sotatercept) for the treatment of adults with PAH to increase exercise capacity, improve World Health Organization (WHO) functional class, and reduce the risk of clinical worsening events.

As discussed above, sotatercept is a recombinant fusion protein consisting of the wild-type extracellular domain (ECD) of human activin receptor type IIA (SEQ ID NO: 1) (ActRIIA, encoded by ACVR2A) fused by a short linker TGGG (SEQ ID NO: 2) to a human IgG1 Fc domain,

(SEQ ID NO: 3)
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPVPIEKTISKAKGQPREPQVYTLPPSREEMTKNQVSLTCLV
KGFYPSDIAVEWESNGQPENNYKTTPPVLDSDGSFFLYSKLTVDKSRWQ
QGNVFSCSVMHEALHNHYTQKSLSLSPGK.

The full-length sequence of sotatercept (SEQ ID NO:4) is as follows:

(SEQ ID NO: 4)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

In some embodiments, sotatercept includes a post translational modification, wherein the C-terminal lysine is clipped. The amino acid sequence of such sotatercept variant lacking the C-terminal lysine is shown below, as purified from CHO cell lines

(SEQ ID NO: 5):
(SEQ ID NO: 5)
5):ILGRSETQECLFFNANWEKDRTNQTGVEPCYGDKDKRRHCFATWKN
ISGSIEIVKQGCWLDDINCYDRTDCVEKKDSPEVYFCCCEGNMCNEKFS
YFPEMEVTQPTSNPVTPKPPTGGGTHTCPPCPAPELLGGPSVFLFPPKP
KDTLMISRTPEVTCVVVDVSHEDPEVKFNWYVEGVEVHNAKTKPREEQY
NSTYRVVSVLTVLHQDWLNGKEYKCKVSNKALPVPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPG

### Cell-Based Potency Assay

Described herein are in vitro cell-based potency assays to determine the potency of, or monitor the potency over time of, an ActRIIA-Fc fusion protein, such as sotatercept, or a pharmaceutical composition containing ActRIIA-Fc fusion protein, such as sotatercept.

In certain embodiments, the methods for determining the relative in vitro potency of an ActRIIA-Fc fusion protein or a composition comprising the ActRIIA-Fc fusion protein described herein comprise:
(i) adding a TGF-β super family ligand to a first portion and a second portion of a cell suspension comprising cells that endogenously express the receptors required for TGF-β super family ligand activation, wherein the cells are engineered to stably express a firefly luciferase (FFLuc) reporter gene, wherein the TGF-β super family ligand is selected from Activin A, Activin B and GDF11;
(ii) adding a test sample comprising a known amount of the ActRIIA-Fc fusion protein to the first portion of the cell suspension to make a test mixture;
(iii) adding a reference sample comprising the ActRIIA-Fc fusion protein to the second portion of the cell suspension to make a reference mixture;
(iv) adding a chemiluminescent substrate solution to the test mixture and the reference mixture;
(v) measuring the luminescence of the test mixture and the reference mixture; and
(vi) determining the relative potency by comparing the luminescence of the test mixture and the reference mixture.

Examples of ActRIIA-Fc fusion proteins for which the described cell-based potency assays can be used are described above. In certain embodiments, the ActRIIA-Fc fusion protein is sotatercept.

For example, in certain embodiments the cell-based potency assay comprises the steps of adding a TGF-β super family ligand to a first portion and a second portion of a cell suspension; (ii) adding a test sample comprising sotatercept to the first portion of the mixture of the cell suspension and TGF-β super family ligand to make a test mixture; (iii) adding a reference sample comprising sotatercept to the second portion of the mixture of the cell suspension and TGF-β super family ligand to make a reference mixture; (iv) adding a chemiluminescent substrate solution to the test mixture and the reference mixture; (v) measuring the luminescence of the test mixture and the reference mixture; and (vii) determining the relative potency by comparing the luminescence of the test mixture and the reference mixture.

Suitable TGF-β super family ligand that can be used in the cell-based assays described here include, but are not limited to, TGF-β proteins, Bone Morphogenetic Proteins (BMPs), Growth Differentiation Factors (GDFs), Glial-derived Neurotrophic Factors (GDNFs), Activins, Inhibins, Nodal, Lefty and Mülllerian Inhibiting Substance (MIS). In certain embodiments, the TGF-β super family ligand is Activin A, Activin B or GDF11. In certain embodiments, the TGF-β super family ligand is Activin A.

For example, in certain embodiments the cell-based potency assay comprises the steps of adding activin A to a first portion and a second portion of a cell suspension; (ii) adding a test sample comprising sotatercept to the first portion to make a test mixture; (iii) adding a reference sample comprising sotatercept to the second portion to make a reference mixture; (iv) adding a chemiluminescent substrate solution to the test mixture and the reference mixture; (v) measuring the luminescence of the test mixture and the reference mixture; and (vii) determining the relative potency by comparing the luminescence of test mixture and the reference mixture.

Cell lines that can be included in the cell suspension and used in the cell-based potency assay include any cell lines that are capable of expressing a TGF-β superfamily ligand and can be transfected with the TGF-β superfamily ligand responsive luciferase reporter plasmid. In certain embodiments, the cell lines used in the cell-based potency assay are A204 (CAGA)₁₂ FFLuc Smad2/3 reporter cell line (“(CAGA)₁₂” disclosed as SEQ ID NO: 23). In some embodiments, the A204 (CAGA)₁₂ FFLuc Smad2/3 reporter cell line (“(CAGA)₁₂” disclosed as SEQ ID NO: 23) can be generated by transfecting human A204 cells (ATCC HTB-82) with a TGF-β family responsive firefly luciferase reporter plasmid containing a hygromycin resistance gene, selecting with Hygromycin B, and single-cloning by limiting dilution. In some embodiments, the A204 (CAGA)₁₂ FFLuc Smad2/3 reporter cell line (“(CAGA)₁₂” disclosed as SEQ ID NO: 23) can be generated by transfecting human A204 cells (ATCC HTB-82) with an Activin A responsive firefly luciferase reporter plasmid (pGL3 (CAGA)₁₂ (SEQ ID NO: 23)) containing a hygromycin resistance gene, selecting with Hygromycin B, and single-cloning by limiting dilution. In certain embodiments, the cell line comprises cells that endogenously express the receptors required for TGF-β super family ligand activation and are engineered to stably express a firefly luciferase (FFLuc) reporter gene under a (CAGA)₁₂ promoter (SEQ ID NO: 23) (Smad2/3 reporter cell line). In certain embodiments, the cell line comprises cells that endogenously express the receptors required for TGF-β super family ligand activation and are engineered to stably express a firefly luciferase (FFLuc) reporter gene under a (CAGACA) n promoter (SEQ ID NO: 24) (Smad2/3 reporter cell line), wherein n is 1, 2, 3, 4, 5, 6, 7, 8, 9, 10, 11 or 12.

In certain embodiments, cells are suspended or resuspended in assay medium and plated on cell culture plates. In certain embodiments, the assay medium comprises one or more of McCoy's 5A, Opti-MEM™ medium (Gibco), fetal bovine serum, penicillin-streptomycin, penicillin-streptomycin, L-Glutamine, or Hygromycin B. In certain embodiments, the assay medium is Opti-MEM™ medium (Gibco). In certain embodiments, the assay medium is Opti-MEM™ medium (Gibco) supplemented with fetal bovine serum. In certain embodiments, the assay medium comprises McCoy's 5A, Opti-MEM™ medium, fetal bovine serum, penicillin-streptomycin, L-Glutamine and Hygromycin B. In certain embodiments, the assay medium comprises a 3:1 ratio of McCoy's 5A to Opti-MEM™ medium+10% fetal bovine serum+1% penicillin-streptomycin+2 mM L-Glutamine+50 ng/ml Hygromycin B.

In certain embodiments, the cell-based potency assay further comprises seeding a population of cells on a cell culture plate. In certain embodiments, the cell-based potency assay comprises the steps of seeding cells on a cell culture plate; adding Activin A to the cells; adding a sotatercept test sample to a portion of the seeded cells and Activin A and adding a sotatercept reference sample to another portion of the seeded cells and Activin A; adding a luminescent substrate to the cells with Activin A and the test sample and to the cells with Activin A and the reference sample; detecting luminescence of the test samples and the reference samples; and comparing the luminescence between the sotatercept reference samples and the sotatercept test samples.

In one embodiment, the cell culture plate comprises at least 6, 12, 24, 48, 96, 384 or 1536 wells. In one embodiment, the plate comprises at least 12 wells. In another embodiment, the plate comprises at least 24 wells. In another embodiment, the plate comprises at least 48 wells. In another embodiment, the plate comprises at least 96 wells. In another embodiment, the plate comprises at least 384 wells. In another embodiment, the plate comprises at least 1536 wells. In one embodiment, the plate is a 96-well plate.

In embodiments where the cell culture plate is a 96 well plate and the cell culture plate is not coated, the method further comprises seeding the cell culture plate with about 1.1×10⁵ cells to about 1.4×10⁵ cells per well. In another embodiment, the cell culture plate is seeded with 1.2×10⁵ cells per well.

In embodiments where the cell culture plate is a 96-well plate and the cell culture plate is coated, the method further comprises seeding the cell culture plate at a density of about 15,000 cells per well to 35,000 cells per well. In one embodiment, the wells of the cell culture plate are seeded with 20,000 cells per well to 30,000 cells per well. In other embodiment, the wells of the cell culture plate are seeded with about 20,000 cells per well. In another embodiment, the wells are seeded with 30,000 cells per well.

In one embodiment of the method, the wells of the cell culture plate do not contain a coating. In another embodiment, the wells of the cell culture plate are coated. In a further embodiment, the wells of the cell culture plate are coated with collagen or lysine. In one embodiment, the cells are coated with collagen. In another embodiment, the cells are coated with lysine.

In certain embodiments of the cell-based assay described herein, A204 (CAGA)₁₂ FFLuc Smad2/3 reporter cells (“(CAGA)₁₂” disclosed as SEQ ID NO: 23) are resuspended in assay medium [Opti-MEM™ medium (Gibco) supplemented with 1% FBS (Hyclone)] and plated in 96-well tissue culture-treated plates.

Once the cells are plated, in certain embodiments the cells are incubated for a period of time prior to the addition of a TGF-β super family ligand and/or an ActRIIA-Fc fusion protein. In certain embodiments, once the cells are plated, the cells are incubated for at least ten minutes. In certain embodiments, once the cells are plated, the cells are incubated for about 10 minutes, 20 minutes, 30 minutes, 40 minutes, 50 minutes or 60 minutes. In certain embodiments, once the cells are plated, the cells are incubated for at least one hour. In certain embodiments, once the cells are plated, the cells are incubated for about one hour, two hours, three hours, four hours or longer. In certain embodiments, once the cells are plated, the cells are incubated between 1-4 hours.

Cells should be incubated under conditions recommended by the cell assay medium. In certain embodiments, the cells, once in the cell assay medium, are incubated at a temperature between 30-40° C. In certain embodiments, the cells, once in the cell assay medium, are incubated at about 30° C., 31° C., 32° C., 33° C., 34° C., 35° C., 36° C., 37° C., 38° C., 39° C., or 40° C. In certain embodiments, the cells, once in the cell assay medium, are incubated at about 37° C. In certain embodiments, the cells, once in the cell assay medium, are incubated at 37° C.

In certain embodiments, the cells, once in the cell assay medium, are incubated in an environment of 5-10% CO₂. In certain embodiments, the cells, once in the cell assay medium, are incubated in an environment of 5, 6, 7, 8, 9 or 10% of CO₂. In certain embodiments, the cells, once in the cell assay medium, are incubated at about 5% CO₂. In certain embodiments, the cells, once in the cell assay medium, are incubated in an environment of 5% CO₂.

In the cell-based assays described herein, once the cells have been incubated, ActRIIA-Fc fusion protein test and/or ActRIIA-Fc fusion protein reference samples can be added to the cells.

ActRIIA-Fc fusion protein test and/or ActRIIA-Fc fusion protein reference samples can be independently prepared. In certain embodiments, dilutions of ActRIIA-Fc fusion protein test and ActRIIA-Fc fusion protein reference samples can be independently prepared, added in duplicate to dilution plates, and then serially diluted in assay medium. In certain embodiments, ActRIIA-Fc fusion protein test and/or ActRIIA-Fc fusion protein reference samples are diluted to a concentration between 10-100 ng/mL. In certain embodiments, ActRIIA-Fc fusion protein test and/or ActRIIA-Fc fusion protein reference samples are diluted to a concentration between 20-80 ng/mL. In certain embodiments, ActRIIA-Fc fusion protein test and/or ActRIIA-Fc fusion protein reference samples are diluted to a concentration between 10-50 ng/ml. In certain embodiments, ActRIIA-Fc fusion protein test and/or ActRIIA-Fc fusion protein reference samples are diluted to a concentration of about 50 ng/mL. In certain embodiments, ActRIIA-Fc fusion protein test and/or ActRIIA-Fc fusion protein reference samples are diluted to a concentration of 50 ng/mL. In certain embodiments, ActRIIA-Fc fusion protein test and/or ActRIIA-Fc fusion protein reference samples are diluted to a concentration of 50 ng/ml of ActRIIA-Fc fusion protein (R&D Systems) in Opti-MEM™+1% FBS.

In the cell-based assays described herein, once the cells have been incubated, sotatercept test and/or sotatercept reference samples can be added to the cells. Dilutions of sotatercept test and reference samples can be independently prepared, added in duplicate to dilution plates, and then serially diluted in assay medium. In certain embodiments, sotatercept test and/or sotatercept reference samples are diluted to a concentration between 10-100 ng/ml. In certain embodiments, sotatercept test and/or sotatercept reference samples are diluted to a concentration between 20-80 ng/mL. In certain embodiments, sotatercept test and/or sotatercept reference samples are diluted to a concentration between 10-50 ng/mL. In certain embodiments, sotatercept test and/or sotatercept reference samples are diluted to a concentration of about 50 ng/ml. In certain embodiments, ActRIIA-Fc fusion protein test and/or ActRIIA-Fc fusion protein reference samples are diluted to a concentration of 50 ng/mL. In certain embodiments, sotatercept test and/or sotatercept reference samples are diluted to a concentration of 50 ng/ml of Activin A protein (R&D Systems) in Opti-MEM™+1% FBS.

In certain embodiments, once the ActRIIA-Fc fusion protein test and references samples are transferred to each plate, the samples are incubated for a period of time. In certain embodiments the samples are incubated overnight. In certain embodiments, the samples are incubated for at least 8 hours. In certain embodiments, the samples are incubated between 8-24 hours. In certain embodiments, the samples are incubated for about 8, 9, 10, 11, 12, 13, 14, 15, 16, 17, 18, 19, 20, 21, 22, 23 or 24 hours.

The cell-based potency assay described herein is designed to be completed in 2 days (48 hours) or less. The chosen incubation times for the cells and the samples for the cell-based potency assay described herein should allow the cell-based assay to be completed in 48 hours or less.

In certain embodiments, once the ActRIIA-Fc fusion protein test and references samples are transferred to each plate, the samples should be incubated under conditions recommended by the cell assay medium. In certain embodiments, the cells, once in the cell assay medium, are incubated at a temperature between 30-40° C. In certain embodiments, the cells, once in the cell assay medium, are incubated at about 30° C., 31° C., 32° C., 33° C., 34° C., 35° C., 36° C., 37° C., 38° C., 39° C., or 40° C. In certain embodiments, the cells, once in the cell assay medium, are incubated at about 37° C. In certain embodiments, the cells, once in the cell assay medium, are incubated at 37° C.

In certain embodiments, once the ActRIIA-Fc fusion protein test and references samples are transferred to each plate, the samples are incubated in an environment of 5-10% CO₂. In certain embodiments, the cells, once in the cell assay medium, are incubated in an environment of 5, 6, 7, 8, 9 or 10% of CO₂. In certain embodiments, the cells, once in the cell assay medium, are incubated at about 5% CO₂. In certain embodiments, the cells, once in the cell assay medium, are incubated in an environment of 5% CO₂. In certain embodiments, the diluted sotatercept samples are transferred to each plate and incubated at 37° C., 5% CO₂ for approximately 24 hours.

Once the samples have been incubated, a luminescent substrate is added. In certain embodiments, the luminescent substrate is ONE-Glo™ luciferase reagent (Promega).

Once the luminescent substrate is added, the samples are incubated for a period of time at room temperature. In certain embodiments, the samples are incubated between 5-30 minutes. In certain embodiments, the samples are incubated for about 5, 10, 15, 20, 25 or 30 minutes. In certain embodiments, the samples are incubated between 5-15 minutes. In certain embodiments, the samples are incubated between 10-15 minutes.

After incubation, a luminescence signal can be measured using a high sensitivity luminescence plate reader (Perkin Elmer Envision, Molecular Devices Spectramax L, or Molecular Devices Spectramax i3x).

Also described herein are methods for determining the relative in vitro potency of sotatercept or a composition comprising sotatercept. In certain embodiments the methods for determining the relative in vitro potency of sotatercept or a composition comprising sotatercept comprise (i) adding Activin A to a first portion and a second portion of a cell suspension comprising cells that express the receptors required for Activin A activation and stably express a firefly luciferase (FFLuc) reporter gene under a (CAGA)₁₂ promoter (SEQ ID NO: 23); (ii) adding a test sample comprising sotatercept to the first portion of the mixture of the cell suspension and Activin A to make a test mixture; (iii) adding a reference sample comprising sotatercept to a second portion of the mixture of the cell suspension and Activin A to make a reference mixture; (iv) adding a chemiluminescent substrate solution to the test mixture and the reference mixture; (v) measuring the luminescence of the test mixture and the reference mixture; (vi) comparing the luminescence of test mixture and the reference mixture; and (vii) determining the relative potency by comparing the luminescence of the test mixture and the reference mixture.

Additionally, described herein are release assays for testing a batch of a pharmaceutical composition comprising an ActRIIA-Fc fusion protein, comprising (i) determining the relative in vitro potency of a test mixture of the pharmaceutical composition from the batch according to any of the methods described herein; and (ii) releasing the batch for in vivo use if the relative in vitro potency value determined in step (i) falls within a range of acceptable values.

In certain embodiments, the methods for determining the relative in vitro potency of sotatercept or a composition comprising sotatercept described herein comprise the step of preparing a cell suspension wherein the cell suspension comprises cells that express the receptors required for Activin A activation and stably express a firefly luciferase (FFLuc) reporter gene under a (CAGA)₁₂ promoter (SEQ ID NO: 23). In certain embodiments, the cells in the cell suspension are A204 (CAGA)₁₂ FFLuc Smad2/3 reporter cell line (“(CAGA)₁₂” disclosed as SEQ ID NO: 23). In some embodiments, the A204 (CAGA)₁₂ FFLuc Smad2/3 reporter cell line (“(CAGA)₁₂” disclosed as SEQ ID NO: 23) can be generated by transfecting human A204 cells (ATCC HTB-82) with a TGF-β family responsive firefly luciferase reporter plasmid containing a hygromycin resistance gene, selecting with Hygromycin B, and single-cloning by limiting dilution. In some embodiments, the A204 (CAGA)₁₂ FFLuc Smad2/3 reporter cells (“(CAGA)₁₂” disclosed as SEQ ID NO: 23) can be generated by transfecting human A204 cells (ATCC HTB-82) with an Activin A responsive firefly luciferase reporter plasmid (pGL3 (CAGA)₁₂ (SEQ ID NO: 23)) containing a hygromycin resistance gene, selecting with Hygromycin B, and single-cloning by limiting dilution.

In certain embodiments, the cell suspension comprises cells that express the receptors required for Activin A activation and stably express a firefly luciferase (FFLuc) reporter gene under a (CAGA)₁₂ promoter (SEQ ID NO: 23) (Smad2/3 reporter cell line) and an assay medium.

In certain embodiments, the cell suspension also comprises an assay medium comprising one or more of McCoy's 5A, Opti-MEM™ medium (Gibco), fetal bovine serum, penicillin-streptomycin, penicillin-streptomycin, L-Glutamine, or Hygromycin B. In certain embodiments, the assay medium is Opti-MEM™ medium (Gibco). In certain embodiments, the assay medium is Opti-MEM™ medium (Gibco) supplemented with fetal bovine serum. In certain embodiments, the assay medium comprises McCoy's 5A, Opti-MEM™ medium, fetal bovine serum, penicillin-streptomycin, L-Glutamine, Hygromycin B. In certain embodiments, the assay medium comprises a 3:1 ratio of McCoy's 5A to Opti-MEM™ medium+10% fetal bovine scrum+1% penicillin-streptomycin+2 mM L-Glutamine+50 ng/ml Hygromycin B.

In certain embodiments the methods for determining the relative in vitro potency of sotatercept or a composition comprising sotatercept, comprises seeding a population of cells on a cell culture plate. In one embodiment, the cell culture plate comprises at least 6, 12, 24, 48, 96, 384 or 1536 wells. In one embodiment, the plate comprises at least 12 wells. In another embodiment, the plate comprises at least 24 wells. In another embodiment, the plate comprises at least 48 wells. In another embodiment, the plate comprises at least 96 wells. In another embodiment, the plate comprises at least 384 wells. In another embodiment, the plate comprises at least 1536 wells. In one embodiment, the plate is a 96 well plate.

In embodiments where the cell culture plate is a 96 well plate and the cell culture plate is not coated, the method further comprises seeding the cell culture plate with about 1.1×10⁵ cells to about 1.4×10⁵ cells per well. In another embodiment, the cell culture plate is seeded with 1.2×10⁵ cells per well.

In embodiments where the cell culture plate is a 96 well plate and the cell culture plate is coated, the method further comprises seeding the cell culture plate at a density of about 15,000 cells per well to 35,000 cells per well. In one embodiment, the wells of the cell culture plate are seeded with 20,000 cells per well to 30,000 cells per well. In other embodiment, the wells of the cell culture plate are seeded with about 20,000 cells per well. In another embodiment, the wells are seeded with 30,000 cells per well.

In certain embodiments of the method for determining the relative in vitro potency of sotatercept or a composition comprising sotatercept, the wells of the cell culture plate do not contain a coating. In another embodiment, the wells of the cell culture plate are coated. In a further embodiment, the wells of the cell culture plate are coated with collagen or lysine. In one embodiment, the cells are coated with collagen. In another embodiment, the cells are coated with lysine.

In certain embodiments of the methods for determining the relative in vitro potency of sotatercept or a composition comprising sotatercept described herein comprising the step of preparing a cell suspension wherein the cell suspension comprises cells that express the receptors required for Activin A activation and stably express a firefly luciferase (FFLuc) reporter gene under a (CAGA)₁₂ promoterA204 (SEQ ID NO: 23) (CAGA)₁₂ FFLuc Smad2/3 (“(CAGA)₁₂” disclosed as SEQ ID NO: 23) the reporter cells are suspended of resuspended in an assay medium comprising [Opti-MEM™ medium (Gibco) supplemented with 1% FBS (Hyclone)] and plated in 96-well tissue culture-treated plates.

Once the cells are plated, in certain embodiments the cells are incubated for a period of time prior to the addition of Activin A and/or sotatercept. In certain embodiments, once the cells are plated, the cells are incubated for at least ten minutes. In certain embodiments, once the cells are plated, the cells are incubated for about 10 minutes, 20 minutes, 30 minutes, 40 minutes, 50 minutes or 60 minutes. In certain embodiments, once the cells are plated, the cells are incubated for at least one hour. In certain embodiments, once the cells are plated, the cells are incubated for about one hour, two hours, three hours, four hours or longer. In certain embodiments, once the cells are plated, the cells are incubated between 1-4 hours.

Cells should be incubated under conditions recommended by the cell assay medium. In certain embodiments, the cells, once in the cell assay medium, are incubated at a temperature between 30-40° C. In certain embodiments, the cells, once in the cell assay medium, are incubated at about 30° C., 31° C., 32° C., 33° C., 34° C., 35° C., 36° C., 37° C., 38° C., 39° C., or 40° C. In certain embodiments, the cells, once in the cell assay medium, are incubated at about 37° C. In certain embodiments, the cells, once in the cell assay medium, are incubated at 37° C.

In certain embodiments, the cells, once in the cell assay medium, are incubated in an environment of 5-10% CO₂. In certain embodiments, the cells, once in the cell assay medium, are incubated in an environment of 5, 6, 7, 8, 9 or 10% of CO₂. In certain embodiments, the cells, once in the cell assay medium, are incubated at about 5% CO₂. In certain embodiments, the cells, once in the cell assay medium, are incubated in an environment of 5% CO₂.

In certain embodiments, the methods for determining the relative in vitro potency of sotatercept or a composition comprising sotatercept comprise a second step of adding Activin A to a first portion and a second portion of the cell suspension. In certain embodiments, once the cells have been added to the wells of a well plate and have been incubated, Activin A can be added to the wells with the incubated cells.

In certain embodiments, the methods for determining the relative in vitro potency of sotatercept or a composition comprising sotatercept comprise a third and fourth step of adding a test sample comprising sotatercept to the first portion of the mixture of the cell suspension and Activin A to make a test mixture; and adding a reference sample comprising sotatercept to a second portion of the mixture of the cell suspension and Activin A to make a reference mixture. In certain embodiments, once the cells have been added to the wells of a well plate and have been incubated and, Activin A has been added to the wells with the incubated cells, a test mixture comprising sotatercept is added to a portion of those cells and reference mixture of sotatercept is added to another portion of those cells.

Dilutions of sotatercept test and reference samples can be independently prepared, added in duplicate to dilution plates, and then serially diluted in assay medium. In certain embodiments, sotatercept test and/or sotatercept reference samples are diluted to a concentration between 10-100 ng/mL. In certain embodiments, sotatercept test and/or sotatercept reference samples are diluted to a concentration between 20-80 ng/mL. In certain embodiments, sotatercept test and/or sotatercept reference samples are diluted to a concentration between 10-50 ng/ml. In certain embodiments, sotatercept test and/or sotatercept reference samples are diluted to a concentration of 50 ng/mL. In certain embodiments, sotatercept test and/or sotatercept reference samples are diluted to a concentration of 50 ng/mL of Activin A protein (R&D Systems) in Opti-MEM™+1% FBS.

In certain embodiments, once the sotatercept test and references samples are transferred to each plate, the samples are incubated for a period of time. In certain embodiments the samples are incubated overnight. In certain embodiments, the samples are incubated for at least 8 hours. In certain embodiments, the samples are incubated between 8-24 hours. In certain embodiments, the samples are incubated for about 8, 9, 10, 11, 12, 13, 14, 15, 16, 17, 18, 19, 20, 21, 22, 23 or 24 hours.

The cell-based potency assays described herein are designed to be completed in 2 days (48 hours) or less. The chosen incubation times for the cells and the samples for the cell-based potency assay described herein should allow the cell-based assay to be completed in 48 hours or less.

In certain embodiments, once the sotatercept test and references samples are transferred to each plate, the samples should be incubated under conditions recommended by the cell assay medium. In certain embodiments, the cells, once in the cell assay medium, are incubated at a temperature between 30-40° C. In certain embodiments, the cells, once in the cell assay medium, are incubated at a temperature of about 30° C., 31° C., 32° C., 33° C., 34° C., 35° C., 36° C., 37° C., 38° C., 39° C., or 40° C. In certain embodiments, the cells, once in the cell assay medium, are incubated at about 37° C. In certain embodiments, the cells, once in the cell assay medium, are incubated at 37° C.

In certain embodiments, once the sotatercept test and references samples are transferred to each plate, the samples are incubated in an environment of 5-10% CO₂. In certain embodiments, the cells, once in the cell assay medium, are incubated in an environment of 5, 6, 7, 8, 9 or 10% of CO₂. In certain embodiments, the cells, once in the cell assay medium, are incubated at about 5% CO₂. In certain embodiments, the cells, once in the cell assay medium, are incubated in an environment of 5% CO₂. In certain embodiments, the diluted sotatercept samples are transferred to each plate and incubated at 37° C., 5% CO₂ for approximately 24 hours.

In certain embodiments of the methods for determining the relative in vitro potency of sotatercept or a composition comprising sotatercept described herein, the method comprises a fifth step of adding a chemiluminescent substrate solution to the test mixture and the reference mixture. In certain embodiments, the luminescent substrate is ONE-Glo™ luciferase reagent (Promega).

Once the luminescent substrate is added, the samples are incubated for a period of time at room temperature. In certain embodiments, the samples are incubated between 5-30 minutes. In certain embodiments, the samples are incubated for about 5, 10, 15, 20, 25 or 30 minutes. In certain embodiments, the samples are incubated between 5-15 minutes. In certain embodiments, the samples are incubated between 10-15 minutes.

In certain embodiments of the methods for determining the relative in vitro potency of sotatercept or a composition comprising sotatercept comprises a sixth step comprising measuring the luminescence of the test mixture and the reference mixture.

After incubation, a luminescence signal can be measured using a high sensitivity luminescence plate reader (Perkin Elmer Envision, Molecular Devices Spectramax L, or Molecular Devices Spectramax i3x).

In some embodiments, the method is performed on a series of dilutions of the test sample of the pharmaceutical composition and a series of dilutions of the reference sample of the pharmaceutical composition and a dose-response curve for each of the test samples and reference samples is generated as described herein. The dose responses curves of the test and reference samples can then be compared to determine the relative potency of the test sample of the pharmaceutical composition. In certain embodiments, the relative in vitro potency is calculated by comparing the EC50 of the test sample and the reference sample. In some embodiments, the relative in vitro potency is calculated using the formula:

$$( EC 50 reference standard / EV 50 test sample ) * 100$$

Also described herein are processes for releasing or accepting a batch of a pharmaceutical composition comprising an ActRIIA-Fc fusion protein, comprising: (i) determining the relative in vitro potency of a test mixture of the pharmaceutical composition from the batch according to any of the methods described herein; and (ii) releasing further pharmaceutical compositions from the batch for in vivo use if the results of step (i) indicate an acceptable relative in vitro potency value.

Also provided herein is a process for releasing or accepting a batch of sotatercept or a composition comprising sotatercept, the process comprising determining the relative in vitro potency of a test sample of the pharmaceutical composition from the batch according to any of the embodiment described above, and releasing further a process for analyzing a batch of sotatercept or a composition comprising sotatercept, comprising determining the relative in vitro potency of a test sample of sotatercept or a composition comprising sotatercept from the batch according to the method described above; and releasing further pharmaceutical compositions from the batch for in vivo use if the results determined relative in vitro potency of the test sample indicate an acceptable relative in vitro potency value.

In some embodiments, the process is performed on a series of dilutions of the test sample of the pharmaceutical composition from the batch and a series of dilutions of the reference sample of the pharmaceutical composition and a dose-response curve for each of the test samples and reference samples is generated as described herein. The dose responses curves of the test and reference samples can then be compared to determine the relative potency of the test sample of the pharmaceutical composition. In certain embodiments, the relative in vitro potency is calculated by comparing the EC50 of the test sample and the reference sample. In some embodiments, the relative in vitro potency is calculated using the formula:

$$( EC 50 reference standard / EV 50 test sample ) * 100$$

In one embodiment of the above process, the acceptable relative in vitro potency value is between 50% and 200%. In one embodiment of the above process, the acceptable relative in vitro potency value is between 70% and 135%. In another embodiment, the acceptable relative in vitro potency is 50% or greater.

### EXAMPLES

The cell-based assay examples described herein reflect the proposed mechanism of action of sotatercept. Sotatercept functions as a ligand trap for members of the TGF-β superfamily such as GDF-11 and Activin A, which normally bind to ActRIIA on the cell surface. These ligands preferentially activate Smad2/3 signaling, which leads to a variety of downstream events including pulmonary vascular remodeling, leading to PAH. Sotatercpet prevents the binding of these ligands to ActRIla and thus prevents the induction of overproliferative Smad2/3 signaling.

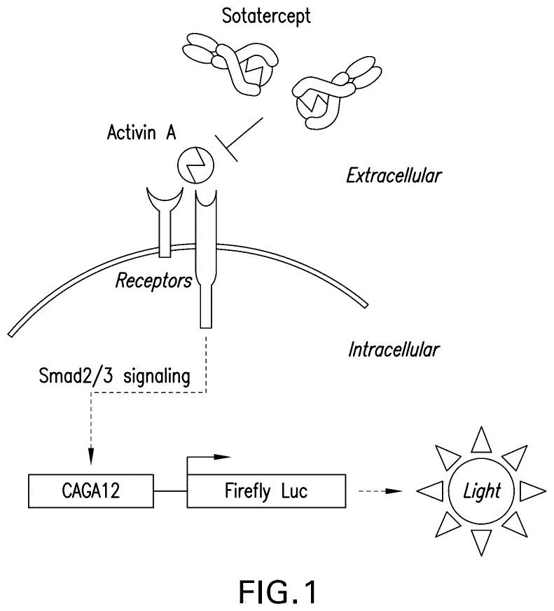

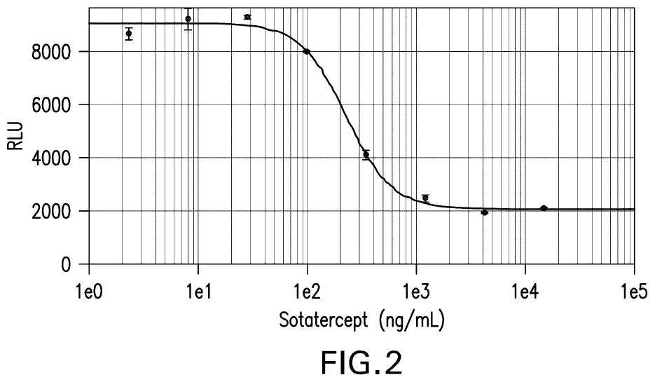

The A204 (CAGA)₁₂ FFLuc cell line (“(CAGA)₁₂” disclosed as SEQ ID NO: 23) naturally expresses ActRIla receptors and was additionally engineered to express a Smad2/3-dependent luciferase reporter ((CAGA)₁₂-FFLuc). Smad2/3 signaling is activated in this cell line upon addition of exogenous Activin A ligand and is detected by the expression of luciferase (FIG. 1). The ability of sotatercept to trap the exogenous ligand and subsequently prevent the induction of the Smad2/3 signaling is determined by detecting the inhibition of the luminescence signal. The luminescence signal, proportional to inhibition of Smad2/3 signaling at different dilutions of sotatercept, is quantified using a luminescence plate reader (FIG. 2). Potency is expressed as a percentage relative to reference material.

### Example 1

### Generation of a A204 Cell Line Stably Expressing (CAGA)₁₂-Firefly Luciferase

A cell line was generated which stably expressed a Smad2/3 reporter, (CAGA)₁₂-firefly luciferase ((CAGA)₁₂-FFLuc). The (CAGA)₁₂ sequence (SEQ ID NO: 23) consists of repetitions of CAGA boxes, which are elements which mediate TGF-β family transcriptional induction (Dennler et al., 1998). The (CAGA)₁₂-FFLuc construct was transduced and integrated into human A204 cells, which endogenously express ActRIIA receptor, via selection with hygromycin. A stable single clone was identified and subsequently banked to use for further assay development. The new stable cell line was given the name A204 (CAGA)₁₂ FFLuc (“(CAGA)₁₂” disclosed as SEQ ID NO: 23).

### Example 2

### Method Optimization

Extensive method optimization was performed to determine the optimal cell culture growth conditions for the A204 (CAGA)₁₂ FFLuc cell line (“(CAGA)₁₂” disclosed as SEQ ID NO: 23), concentration of ligand to use, assay incubation time, cell seeding density, and sotatercept dilution series. The results of the method optimization are summarized in Table 1. Parameters were chosen to improve QC friendliness and robustness of the assay. Note that Activin A was chosen as the representative TGF-β superfamily ligand to use in assays disclosed herein. However, this assay can be used with other TGF-β superfamily ligands, such as GDF11.

TABLE 1
Summary of re-developed functional cell-based assay optimization

| Parameter | Optimized Result | |
|---|---|---|
| Cell culture medium | 3:1 ratio of McCoy's 5A to Opti-MEM ™ | |
| composition | medium + 10% fetal bovine serum + 1% | |
| | penicillin-streptomycin + 2 mM L-Glutamine + | |
| | 50 ng/mL Hygromycin B | |
| Activin A concentration | 50 ng/mL Activin A, diluted in Opti- | |
| | MEM ™ + 1% FBS | |
| Assay incubation time | 24 ± 2 | hours |
| Cell seeding density | 20,000 | cells/well |

| | |
|---|---|
| Sotatercept dilution | 1 to 3.5-fold with 15 ng/mL sotatercept as |
| series | starting concentration |

As a result of the method optimization work, the sotatercept potency release assay is easy to run in a QC space, uses few critical reagents, and requires only two days to run. Smad2/3 signaling is activated in the A204 (CAGA)₁₂ FFLuc stable cell line (“(CAGA)₁₂” disclosed as SEQ ID NO: 23) upon addition of exogenous Activin A, and is detected by the expression of luciferase. The ability of sotatercept to trap the ligand and subsequently prevent the induction of the Smad2/3 signaling is determined by detecting the inhibition of the luminescence signal. The luminescence signal, proportional to inhibition of Smad2/3 signaling at different dilutions of sotatercept, is quantified using a luminescence plate reader. Potency is expressed as a percentage relative to reference material.

### Cell Culture Medium Composition

An experiment was conducted to assess if 1) alternate base media other than McCoy's 5A (i.e., Iscove's Modified Dulbecco's Medium (IMDM) or Dulbecco modified eagle medium (DMEM)) improved cell doubling time, and if 2) addition of assay medium to the various based mediums improved cell doubling time. The results showed that the cells have a shorter doubling time when maintained in either McCoy's 5A or IMDM compared to DMEM, and that when assay medium (Opti-MEM™+1% FBS) is introduced to any of the three base media, the doubling time is significantly shortened, irrespective of base medium.

Based on these results, the final cell culture medium for maintenance of the cell line was adjusted to be a 3:1 ratio of McCoy's 5A to Opti-MEM™ in order to optimize cell growth.

### Activin A Concentration

An Activin A titration was performed to assess the Activin A concentration needed to maximize the luminescence signal in the Smad2/3 reporter A204 (CAGA)₁₂ FFLuc cell line (“(CAGA)₁₂” disclosed as SEQ ID NO: 23). Activin A from two different vendors was diluted in two different types of assay medium (Opti-MEM™+1% FBS or McCoy's 5A+1% FBS), serially diluted, and added to differing seeding density of cells (5,000, 10,000, or 20,000 cells/well) for approximately 24 hours, after which luminescence signal was detected using a Perkin Elmer Envision plate reader.

The results showed that the EC50 values from either vendor were similar (and do not change significantly based on cell seeding density), that Opti-MEM™+1% FBS is a superior assay medium compared to McCoy's 5A+1% FBS due to less shallow slope, and that increasing cell seeding density increased the overall observed signal. Additionally, an Activin A concentration between 50-100 ng/ml is needed to saturate the luminesence signal in these cells.

A concentration of 50 ng/ml Activin A, diluted in Opti-MEM™+1% FBS as assay medium, was chosen for the final method procedure.

### Cell Seeding Density

Various cell seeding densities were tested. Cells were seeded at 7,500, 10,000, or 15,000 cells/well in a single assay plate, incubated with 50 ng/mL Activin A and serially-diluted sotatercept for approximately 24 hours. Luminescence signal was detected with a Molecular Devices Spectramax L plate reader.

The results showed, that while the assay window (A/D) does not significantly change when cells are seeded at differing densities, the total luminescence signal (RLU) does increase with increasing cell density.

While this experiment demonstrated 10,000 cells/well is sufficient for use in the cell-based assay, in practice this led to significant cell waste as the cell line was minimally maintained in a single 75 cm2 flask, which yielded on average ˜10 million cells. Using 20,000 cells/well maximized the cell seeding density for a 3-plate assay setup (when using a single 75 cm2 flask) while still maintaining good assay performance and not overgrowing after an overnight incubation.

Ultimately, a cell seeding density of 20,000 cells/well was chosen for the final method procedure.

Activin A/Sotatercept Incubation Time with Cells

To assess whether the assay procedure could be shortened to a single day, a shorter drug incubation time (6 hours) was tested. 10,000 cells/well were plated and 100 ng/mL Activin A+serially-diluted sotatercept was added either 24 hours or 6 hours prior to measuring luminescence on a Perkin Elmer Envision plate reader. The results showed that the luminescence signal and assay window are increased when the cells are incubated with drug for 24 hours vs. 6 hours.

A 24±2 hours incubation time was chosen for the final assay procedure.

### Sotatercept Dilution Scheme

An optimal dilution scheme was determined by testing various fold-dilutions at either 10 or 15 ng/mL starting sotatercept concentration with 50 ng/mL Activin A and measuring luminescence approximately 24 hours later on a Molecular Devices Spectramax L plate reader.

A 1 to 3.5-fold dilution series of 15 ng/mL starting sotatercept concentration was chosen for the final method procedure.

### Example 3

### In Vitro Cell-Based Potency Assay

### General Assay Conditions:

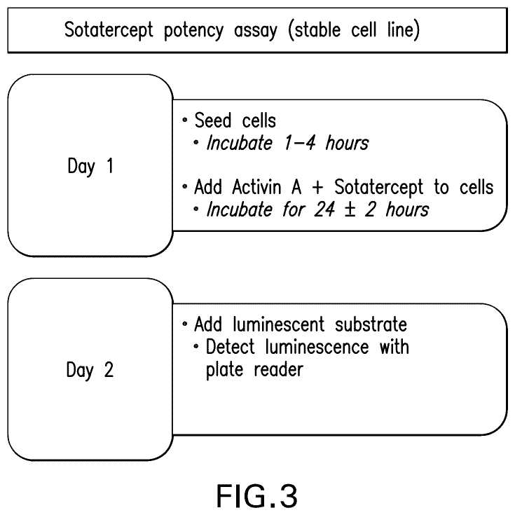

A general schematic of the assay is set forth in FIG. 3. Cell Line and Cell Culture

The A204 (CAGA)₁₂ FFLuc Smad2/3 reporter cell line (“(CAGA)₁₂” disclosed as SEQ ID NO: 23) was generated by transfecting human A204 cells (ATCC HTB-82) with a TGF-β family responsive firefly luciferase reporter plasmid (pGL3 (CAGA)₁₂ (SEQ ID NO: 23)) containing a hygromycin resistance gene, selecting with Hygromycin B, and single-cloning by limiting dilution.

A204 (CAGA)₁₂ FFLuc cells (“(CAGA)₁₂” disclosed as SEQ ID NO: 23) were cultured at 37° C., 5% CO₂ in McCoy's 5A (Gibco) supplemented with 25% Opti-MEM™ (Gibco), 10% fetal bovine serum (FBS) (HyClone), 2 mM L-Glutamine (Gibco), 50 ng/ml Hygromycin B (Gibco), 100 units/mL of penicillin and 100 ng/ml of streptomycin (Gibco).

Parental A204 cells were cultured at 37° C., 5% CO₂ in McCoy's 5A (Gibco) supplemented with 10% FBS (ATCC).

A204 (CAGA)₁₂ FFLuc Smad2/3 reporter cells (“(CAGA)₁₂” disclosed as SEQ ID NO: 23) were resuspended in assay medium [Opti-MEM™ medium (Gibco) supplemented with 1% FBS (Hyclone)] and plated in opaque white 96-well tissue culture-treated plates. Dilutions of sotatercept test and reference samples were independently prepared, added in duplicate to dilution plates, and then serially diluted in assay medium. 50 ng/ml of Activin A protein (R&D Systems) and the serially diluted sotatercept were transferred to each plate and incubated at 37° C., 5% CO₂. After approximately 24 hours, ONE-Glo™ luciferase reagent (Promega), reconstituted according to manufacturer's instruction, was added to all wells. After a 10-15 minute incubation at room temperature, the luminescence signal was measured using a high sensitivity luminescence plate reader (Perkin Elmer Envision, Molecular Devices Spectramax L, or Molecular Devices Spectramax i3x).

The data was fit to a four-parameter logistic (4-PL) model using SoftMax Pro software (Molecular Devices). The potency of each sample is expressed as the percent (%) potency relative to concurrently analyzed reference material after performing parallel line analysis, i.e., [IC50Reference/IC50Test Sample]*100%. The final reported potency result is determined as the geometric mean of three individual potency values obtained from three independently handled assay plates.

Table of Sequences

| | |
|---|---|
| SEQ ID NO: 1 | ILGRSETQECLFFNANWEKDRTNQTGVEPCYGDKDKRRHCFAT |
| (Wild-type | WKNISGSIEIVKQGCWLDDINCYDRTDCVEKKDSPEVYFCCCEG |
| extracellular | NMCNEKFSYFPEMEVTQPTSNPVTPKPP |
| domain (ECD) of | |
| human activin | |
| receptor type IIA) | |
| SEQ ID NO: 2 | TGGG |
| (Linker) | |
| SEQ ID NO: 3 | THTCPPCPAPELLGGPSVFLFPPKPKDTLMISRTPEVTCVVVDVS |
| (Human IGg1 Fc | HEDPEVKFNWYVDGVEVHNAKTKPREEQYNSTYRVVSVLTVL |
| Domain) | HQDWLNGKEYKCKVSNKALPVPIEKTISKAKGQPREPQVYTLPP |
| | SREEMTKNQVSLTCLVKGFYPSDIAVEWESNGQPENNYKTTPPV |
| | LDSDGSFFLYSKLTVDKSRWQQGNVFSCSVMHEALHNHYTQKS |
| | LSLSPGK |
| SEQ ID NO: 4 | ILGRSETQECLFFNANWEKDRTNQTGVEPCYGDKDKRRHCFAT |
| (Sotatercept) | WKNISGSIEIVKQGCWLDDINCYDRTDCVEKKDSPEVYFCCCEG |
| | NMCNEKFSYFPEMEVTQPTSNPVTPKPPTGGGTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPVPIEKTISKAKGQPREPQVYTLPPSREEMTKNQVSLT |
| | CLVKGFYPSDIAVEWESNGQPENNYKTTPPVLDSDGSFFLYSKL |
| | TVDKSRWQQGNVFSCSVMHEALHNHYTQKSLSLSPGK |
| SEQ ID NO: 5 | ILGRSETQECLFFNANWEKDRTNQTGVEPCYGDKDKRRHCFAT |
| (Sotatercept minus | WKNISGSIEIVKQGCWLDDINCYDRTDCVEKKDSPEVYFCCCEG |
| the C terminal | NMCNEKFSYFPEMEVTQPTSNPVTPKPPTGGGTHTCPPCPAPELL |
| lysine) | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPVPIEKTISKAKGQPREPQVYTLPPSREEMTKNQVSLT |
| | CLVKGFYPSDIAVEWESNGQPENNYKTTPPVLDSDGSFFLYSKL |
| | TVDKSRWQQGNVFSCSVMHEALHNHYTQKSLSLSPG |
| SEQ ID NO: 12 | MGAAAKLAFAVFLISCSSGAILGRSETQECLFFNANWEKDRTNQ |
| (Canonical human | TGVEPCYGDKDKRRHCFATWKNISGSIEIVKQGCWLDDINCYDR |
| ActRIIA precursor | TDCVEKKDSPEVYFCCCEGNMCNEKFSYFPEMEVTQPTSNPVTP |
| protein sequence) | KPPYYNILLYSLVPLMLIAGIVICAFWVYRHHKMAYPPVLVPTQ |
| | DPGPPPPSPLLGLKPLQLLEVKARGRFGCVWKAQLLNEYVAVKI |
| | FPIQDKQSWQNEYEVYSLPGMKHENILQFIGAEKRGTSVDVDL |
| | WLITAFHEKGSLSDFLKANVVSWNELCHIAETMARGLAYLHEDI |
| | PGLKDGHKPAISHRDIKSKNVLLKNNLTACIADFGLALKFEAGK |
| | SAGDTHGQVGTRRYMAPEVLEGAINFQRDAFLRIDMYAMGLVL |
| | WELASRCTAADGPVDEYMLPFEEEIGQHPSLEDMQEVVVHKKK |
| | RPVLRDYWQKHAGMAMLCETIEECWDHDAEARLSAGCVGERI |
| | TQMQRLTNIITTEDIVTVVTMVTNVDFPPKESSL |
| SEQ ID NO: 13 | ILGRSETQECLFFNANWEKDRTNQTGVEPCYGDKDKRRHCFAT |
| (Wild-type | WKNISGSIEIVKQGCWLDDINCYDRTDCVEKKDSPEVYFCCCEG |
| extracellular | NMCNEKFSYFPEM |
| domain (ECD) of | |
| human activin | |
| receptor type IIA | |
| with the “tail” | |
| deleted (a Δ15 | |
| sequence) | |
| SEQ ID NO: 14 | atgggagctgctgcaaagttggcgtttgccgtctttcttatctcctgttc |
| (Nucleic acid | ttcaggtgctatacttggtagatcagaaactcaggagtgtcttttcttta |
| sequence encoding | atgctaattgggaaaaagacagaaccaatcaaactggtgttgaaccgtgt |
| the human ActRIIA | tatggtgacaaagataaacggcggcattgttttgctacctggaagaatat |
| precursor protein) | ttctggttccattgaaatagtgaaacaaggttgttggctggatgatatca |
| | actgctatgacaggactgattgtgtagaaaaaaaagacagccctgaagta |
| | tatttttgttgctgtgagggcaatatgtgtaatgaaaagttttcttattt |
| | tccggagatggaagtcacacagcccacttcaaatccagttacacctaagc |
| | caccctattacaacatcctgctctattccttggtgccacttatgttaatt |
| | gcggggattgtcatttgtgcattttgggtgtacaggcatcacaagatggc |
| | ctaccctcctgtacttgttccaactcaagacccaggaccacccccacctt |
| | ctccattactaggtttgaaaccactgcagttattagaagtgaaagcaagg |
| | ggaagatttggttgtgtctggaaagcccagttgcttaacgaatatgtggc |
| | tgtcaaaatatttccaatacaggacaaacagtcatggcaaaatgaatacg |
| | aagtctacagtttgcctggaatgaagcatgagaacatattacagttcatt |
| | ggtgcagaaaaacgaggcaccagtgttgatgtggatctttggctgatcac |
| | agcatttcatgaaaagggttcactatcagactttcttaaggctaatgtgg |
| | tctcttggaatgaactgtgtcatattgcagaaaccatggctagaggattg |
| | gcatatttacatgaggatatacctggcctaaaagatggccacaaacctgc |
| | catatctcacagggacatcaaaagtaaaaatgtgctgttgaaaaacaacc |
| | tgacagcttgcattgctgactttgggttggccttaaaatttgaggctggc |
| | aagtctgcaggcgatacccatggacaggttggtacccggaggtacatggc |
| | tccagaggtattagagggtgctataaacttccaaagggatgcatttttga |
| | ggatagatatgtatgccatgggattagtcctatgggaactggcttctcgc |
| | tgtactgctgcagatggacctgtagatgaatacatgttgccatttgagga |
| | ggaaattggccagcatccatctcttgaagacatgcaggaagttgttgtgc |
| | ataaaaaaaagaggcctgttttaagagattattggcagaaacatgctgga |
| | atggcaatgctctgtgaaaccattgaagaatgttgggatcacgacgcaga |
| | agccaggttatcagctggatgtgtaggtgaaagaattacccagatgcaga |
| | gactaacaaatattattaccacagaggacattgtaacagtggtcacaatg |
| | gtgacaaatgttgactttcctcccaaagaatctagtcta |
| SEQ ID NO: 15 | atacttggtagatcagaaactcaggagtgtcttttctttaatgctaattg |
| (Nucleic acid | ggaaaaagacagaaccaatcaaactggtgttgaaccgtgttatggtgaca |
| sequence encoding | aagataaacggcggcattgttttgctacctggaagaatatttctggttcc |
| the processed | attgaaatagtgaaacaaggttgttggctggatgatatcaactgctatga |
| soluble | caggactgattgtgtagaaaaaaaagacagccctgaagtatatttttgtt |
| (extracellular) | gctgtgagggcaatatgtgtaatgaaaagttttcttattttccggagatg |
| human ActRIIA | gaagtcacacagcccacttcaaatccagttacacctaagccaccc |
| polypeptide) | |
| (Linker) | GGG |
| SEQ ID NO: 17 | GGGG |
| (Linker) | |
| SEQ ID NO: 18 | TGGGG |
| (Linker) | |
| SEQ ID NO: 19 | SGGGG |
| (Linker) | |
| SEQ ID NO: 21 | SGGG |
| (Linker) | |
| SEQ ID NO: 22 | GGGGS |
| (Linker) | |
| SEQ ID NO: 23 | CAGACAGACAGACAGACAGACAGACAGACAGACAGACAGAC |
| (CAGA)₁₂ | AGACAGA |
| SEQ ID NO: 24 | (CAGACA)n, wherein n is 1, 2, 3, 4, 5, 6, 7, 8, 9, 10, 11 or 12 |

1. A method for determining the relative in vitro potency of an ActRIIA-Fc fusion protein or a composition comprising the ActRIIA-Fc fusion protein, the method comprising:
(i) adding a TGF-β super family ligand to a first portion and a second portion of a cell suspension comprising cells that endogenously express the receptors required for TGF-β super family ligand activation, wherein the cells are engineered to stably express a firefly luciferase (FFLuc) reporter gene, wherein the TGF-β super family ligand is selected from Activin A, Activin B and GDF11;
(ii) adding a test sample comprising a known amount of the ActRIIA-Fc fusion protein to the first portion of the cell suspension to make a test mixture;
(iii) adding a reference sample comprising the ActRIIA-Fc fusion protein to the second portion of the cell suspension to make a reference mixture;
(iv) adding a chemiluminescent substrate solution to the test mixture and the reference mixture;
(v) measuring the luminescence of the test mixture and the reference mixture; and
(vi) determining the relative potency by comparing the luminescence of the test mixture and the reference mixture.

2. The method of claim 1, wherein the first portion and the second portion of the cell suspension are incubated for at least 10 minutes after adding the TGF-β super family ligand.

3. (canceled)

4. The method of claim 1, wherein the first portion and the second portion of the cell suspension are incubated at a temperature between 30-40° C.

5. The method of claim 1, wherein the first portion and the second portion of the cell suspension are incubated at about 37° C. and about 5% CO₂.

6. The method of claim 1, wherein the test sample is added to the first portion of the cell suspension at the same time as the TGF-β super family ligand.

7. The method of claim 1, wherein the reference sample is added to the second portion of the cell suspension at the same time as the TGF-β super family ligand.

8. The method of claim 1, wherein before step (v) the reference mixture and the test mixture are incubated overnight.

9-19. (canceled)

20. The method of claim 1, further comprising generating a dose response curve for the test mixture and the reference mixture and determining the reference light unit (RLU) of the test mixture and the reference mixture.

21. The method of claim 1, wherein the relative potency is calculated as a percentage of the reference standard RLU using the formula
$$\% Relative potency = ( Creference / Csample ( or control ) ) \times RS assigned potency per COA .$$

22. The method of claim 1, wherein the reporter gene is under the control of a (CAGA)₁₂ promoter (SEQ ID NO: 23) or a CAGACA promoter.

23. (canceled)

24. The method of claim 1, wherein the TGF-β super family ligand is Activin A.

25. The method of claim 1, wherein the ActRIIA-Fc fusion protein comprises an amino acid sequence that is at least 90% identical to SEQ ID NO: 1.

26-28. (canceled)

29. The method of claim 1, wherein the ActRIIA-Fc fusion protein comprises an amino acid sequence that is at least 90% identical to SEQ ID NO: 4.

30-32. (canceled)

33. The method of claim 1, wherein the ActRIIA-Fc fusion protein comprises an amino acid sequences that is at least 90% identical to SEQ ID NO: 5.

34-36. (canceled)

37. The method of claim 1, wherein the ActRIIA-Fc fusion protein comprises sotatercept.

38. A release assay for testing a batch of a pharmaceutical composition comprising an ActRIIA-Fc fusion protein, comprising
(i) determining the relative in vitro potency of a test mixture of the pharmaceutical composition from the batch according to the method of any of claims 1-34; and
(ii) releasing the batch for in vivo use if the relative in vitro potency value determined in step (i) falls within a range of acceptable values.

39. The release assay for testing a batch of a pharmaceutical composition comprising an ActRIIA-Fc fusion protein of claim 38, wherein the relative in vitro potency value is calculated by generating a dose response curve for the test sample and the reference sample and determining the EC50 of the test sample and reference sample.

40. The release assay for testing a batch of a pharmaceutical composition comprising an ActRIIA-Fc fusion protein of claim 38, wherein the relative in vitro potency value is calculated using the formula
$$( IC 50 reference standard / IC 50 test sample ) * 100.$$

41-42. (canceled)

43. The release assay for testing a batch of a pharmaceutical composition comprising an ActRIIA-Fc fusion protein of claim 38, wherein the ActRIIA-Fc fusion protein comprises an amino acid sequence that is at least 90% identical to SEQ ID NO: 1.

44-54. (canceled)

55. The release assay for testing a batch of a pharmaceutical composition comprising an ActRIIA-Fc fusion protein of claim 38, wherein the ActRIIA-Fc fusion protein comprises sotatercept.
